(19) United States
(12) Patent Application Publication
(10) Pub. No.: US 2015/0313986 A1
(43) Pub. Date: Nov. 5, 2015

(54) PRODUCTION OF HETEROLOGOUS POLYSACCHARIDES IN PLANTS

(71) Applicants: THE UNIVERSITY OF MANCHESTER, Manchester (GB); THE UNIVERSITY OF LEICESTER, Leicester (GB)

(72) Inventors: Ian Roberts, Manchester (GB); Peter Andrew, Leicester (GB); Garry Whitelam (deceased), Leicester (GB)

(73) Assignees: THE UNIVERSITY OF LEICESTER, Leicester (GB); THE UNIVERSITY OF MANCHESTER, Manchester (GB)

(21) Appl. No.: 14/650,968

(22) PCT Filed: Dec. 10, 2013
(86) PCT No.: PCT/GB2013/053246

§ 371 Date: Jun. 10, 2015

(30) Foreign Application Priority Data
Dec. 10, 2012 (GB) 1222183.4

(51) Int. Cl. A61K 39/09; C12N 9/10; C08B 37/00; C12P 19/04

(52) U.S. Cl. CPC A61K 39/092; C12P 19/04; C12N 9/1048; C08B 37/006; A61K 2039/517

(57) ABSTRACT

The invention relates to a novel method for producing bacterial or viral saccharides, such as capsular, immunogenic or membrane saccharides, in a plant cell. Also provided plants, plant cells, plant parts, and plant products comprising such a bacterial or viral saccharide. The invention is based upon the novel finding that bacterial enzymes can be used in a plant cell to produce bacterial or viral capsular, immunogenic and/or membrane saccharides.

### SUMMARY

The invention provides a method of producing a bacterial capsular saccharide, a bacterial membrane saccharide and/or a bacterial or viral immunogenic saccharide, in a plant. Also provided are plants, plant parts, plant cells, plant extracts, plant tissue or callus and plant products comprising a bacterial capsular saccharide, a bacterial membrane saccharide and/or a bacterial or viral immunogenic saccharide; and uses of the same, for example, as a vaccine. The invention also provides a bacterial capsular saccharide, a bacterial membrane saccharide and/or a bacterial or viral immunogenic saccharide produced in a plant.

### BACKGROUND

The manipulation of plants to improve characteristics as diverse as flower development, seed development, drought resistance, improved nutrient uptake and improved or modified seed content, has been known for many years using recombinant and non-recombinant techniques. Plants have also been used to produce products originally derived from organisms such as bacteria or fungi. For example, the production of polyalkanoates, such as polyhydroxybutyrate (PHB) or polyhydroxyvalerate (PHV) (see, for example, WO 92/19747), have been carried out in plants.

Transgenic plants are already recognised as a valuable expression system for the production of proteins. The idea was first discussed in 1990 in PCT/US89/03799 which reported the expression of a surface protein of Streptococcus mutans surface protein antigen A (SpaA) in tobacco plants. Although no further details of this study have been reported, it paved the way for further development, including the expression of Hepatitis B virus surface antigen (HbsAg) and enterotoxins such as E. coli heat-labile enterotoxin B subunit (LTB).

Plants have also been used for the production of saccharides. For example, WO96/01904 describes the production of oligosaccharides in a plant cell, by transforming the plant cell with an enzyme capable of converting sucrose into an oligosaccharide. In a similar vein, WO96/06173 describes the genetic manipulation of a plant to increase the amount of stored carbohydrates, such as sucrose, fructose and glucans. The method involves transforming a plant with bacterial enzyme [β]-D-glucosyl transferase, which catalyses the formation of glucan. WO00/9729 describes the production of chitin in plants, by transforming a plant with one or more chitin producing enzymes.

The art has described the use of bacterial enzymes in plants, but this has been limited to the production of bacterial carbohydrates such as dextran. WO 96/06173 describes the production of genetically modified plants which express a Streptococcal glycosylation luciferase to catalyse production of soluble glucans, such as dextran. WO2006/063862 descries the expression of dextransucrose from bacteria (L. mesenterides) in plastids, to alter starch production by providing dextran in plants cells.

Plants have also been modified to alter the production of native polysaccharides, such as starch. This has been achieved in some cases by manipulating the plant polysaccharide pathway. WO03/026389 discloses a method of manipulating plant polysaccharides by altering the polysaccharide synthesis pathway in plants. Specifically, the method reroutes polysaccharides from one biosynthetic pathway in the plant to another in the plant to alter the polysaccharide composition of the plant cells, tissues or organs. Thus, the polysaccharides produced are native plant polysaccharides. Further, U.S. Pat. No. 6,639,126 shows the modification of the physical characteristics of e.g. starch, by modifying pullunase enzymes.

There are a number of commercially and medically important polysaccharides produced by non-plant organisms. For example, pathogenic bacteria commonly produce a thick, mucus capsule of polysaccharide. Such polysaccharides have been isolated from the original organisms and used as vaccines. As polysaccharide encapsulated bacteria are a major cause of morbidity and mortality worldwide (e.g. Streptococcus pneumoniae is responsible for more than 50% of invasive disease worldwide), and vaccination is the only preventative method, successful vaccination programmes are extremely important.

Polysaccharide vaccines are expensive, and this inhibits their use in developing countries. A factor that increases the likelihood of a country introducing a vaccine, such as a polysaccharide vaccine, is its ability to manufacture the vaccine itself.

There are over 90 different serotypes of Streptococcus pneumoniae, each with a different polysaccharide coating. Current vaccines are composed of up to 23 of the most dominant serotypes of a particular geographical area, rendering them less effective in other areas. In areas where they are effective, immunity has led to the emergence of serotypes not in the vaccine. Thus, the pneumococcal vaccines require regular reformulation to offer maximum protection.

The present invention aims to overcome or ameliorate some or all of the problems associated with the prior art.

Thus, in a first aspect the present invention provides a method of producing i) a bacterial capsular saccharide, ii) a bacterial membrane saccharide and/or iii) a bacterial or viral immunogenic saccharide in a host plant cell, the method comprising:

1) providing a plant cell;
2) introducing into the plant cell a heterologous protein or nucleotide sequence which enables the synthesis of said bacterial or viral saccharide in the plant cell;
3) maintaining the host plant cell under suitable conditions for synthesis of said bacterial or viral saccharide; and optionally
4) harvesting the bacterial or viral saccharide from the host plant cell.

The present invention enables for the first time the synthesis of i) a bacterial capsular saccharide, ii) a bacterial membrane saccharide and/or iii) a bacterial or viral immunogenic saccharide in a plant cell, preferably using a bacterial or viral enzyme. Typically, this comprises introducing a heterologous (i.e. non-native to the plant cell) saccharide synthesis step into a plant cell. The method of the invention may comprise comparing a native pathway for synthesis of the bacterial capsular saccharide, bacterial membrane saccharide and/or bacterial or viral immunogenic saccharide with a native plant saccharide biosynthesis pathway to identify any reaction step(s) required in the host plant cell to enable the bacterial or viral saccharide to be produced by the host plant cell. Optionally, the comparison may be used to identify any steps in a native plant saccharide biosynthesis pathway which are superfluous for the production of a bacterial or viral saccharide, or which might prevent or reduce production of a bacterial or viral saccharide (e.g. through competition). Thus, a host plant cell may be exploited for the production of i) a bacterial capsular saccharide, ii) a bacterial membrane saccharide and/or iii) a bacterial or viral immunogenic saccharide by providing in the host plant cell a heterologous protein or nucleotide sequence which is able to provide a reaction step to enable the synthesis of the bacterial or viral saccharide biosynthesis pathway in the host plant cell. Preferably, the modified plant pathway is able to use native plant substrate. Alternatively, heterologous substrate may be used.

The present invention may have particular applicability to the production of vaccines. Transgenic plants offer a solution to problems of cost and local availability of stable vaccines, particularly in third world countries where the greatest burden of vaccine-preventable diseases is found but also where the economy is least able to bear the costs. The ability to produce saccharide vaccines in plants on a large scale introduces economies of scale that can drive down the production costs. The use of microorganisms as a vaccine production system requires expensive fermentation equipment and high levels of quality control to prevent contamination, as well as containing pathogenic organisms which represent a health and safety risk. In contrast, the use of plants for vaccine production is a relatively low-cost solution, opening up the possibility of local production. Producing vaccines in edible plants can reduce costs further by removing the need for down-stream processing. Also edible transgenic plants provide an improved means of delivery of vaccines by providing a system that dispenses with the need for needles. Not only may this be more acceptable to recipients, it may reduce exposure of health care workers to blood-contaminated sharps.

An additional advantage of the use of plant production systems, a traditional source of pharmaceuticals and foodstuffs, relates to safety and downstream processing costs. The cells of edible plants do not harbour human pathogens or contaminants such as pyrogens and endotoxins, and consequently extensive purification procedures can be avoided.

Finally, the predicted financial benefits of plant based production of microbial polysaccharides over the more expensive microbial fermentation systems may increase wider industrial usage of bacterial polysaccharides.

A bacterial capsular or membrane saccharide produced by the present invention may be an immunogenic saccharide.

A method of the first aspect may further comprise:

(i) identifying the molecular structure and/or elucidating the native bacterial or viral saccharide biosynthesis pathway of i) a bacterial capsular saccharide, ii) a bacterial membrane saccharide and/or iii) a bacterial or viral immunogenic saccharide;
(ii) comparing the molecular structure and/or the native bacterial or viral saccharide biosynthesis pathway identified in step i) with the molecular structure of a native plant saccharide and/or a native plant saccharide biosynthesis pathway of the host plant cell, to identify any reaction step(s) required to enable production of the bacterial or viral saccharide in the host plant cell;
(iii) identifying a protein or nucleotide sequence which can provide a reaction step identified in step ii) to enable the production of the bacterial or viral saccharide in the host plant cell.

The method of the invention may also comprise down-regulating a native plant saccharide biosynthesis pathway or reaction step, to enable production of i) a bacterial capsular saccharide, ii) a bacterial membrane saccharide and/or iii) a bacterial or viral immunogenic saccharide in the host plant cell. Such a reaction step may be one which competes with the selected biosynthesis pathway or competes with a reaction step thereof (e.g. competes for substrate) and thus its down regulation may improve the exploitation of the native plant saccharide biosynthesis pathway for the production of the bacterial or viral saccharide.

A native plant saccharide biosynthesis pathway may be one which produces a saccharide which is naturally found in the plant cell. Thus, the pathway may be one which is naturally performed in the host plant cell, i.e. without human intervention. Alternatively, the pathway may be one which has been previously modified in the host plant cell type, for example in an ancestor of the plant. Where a plant cell saccharide biosynthesis pathway has been previously modified, for example, in an ancestor of the cell or plant, the pathway (and saccharide) may be referred to herein as being native to the host plant cell. A native reaction step is a part of a native pathway.

A native pathway for synthesis of a bacterial or viral saccharide as defined herein is a pathway which naturally produces such a bacterial or viral saccharide in a bacterial cell or in a virus-infected cell. Thus, while steps of the pathway may occur in a plant cell, typically one or more reaction steps leading to production of the bacterial or viral saccharide will not be native to the plant cell. Thus, the pathway is native (i.e. naturally occurring) to a cell other than the selected host plant cell. The pathway may be one which has been previously modified, for example in an ancestor cell. Where the bacterial or viral saccharide biosynthesis pathway has been previously modified, for example in an ancestor of the cell, the pathway (and saccharide) are referred to herein as being native to the cell. Most preferably, the bacterial saccharide biosynthesis pathway is bacterial in origin, and more preferably Streptoccocal. Most preferably, the bacterial saccharide biosynthesis pathway is native to Streptococcus pneumoniae.

A bacterial capsular saccharide, a bacterial membrane saccharide and/or a bacterial or viral immunogenic saccharide to be produced by a host plant cell is not native thereto i.e. in nature, it is not produced by, or present in, the selected host plant cell. Thus, it is referred to as being heterologous to the host plant cell.

By viral is meant that it is found in a virus particle or in a cell infected by a virus although it may not be native to the cell when not infected by the virus. Where the saccharide is native to a virus, it is preferably derived from the viral capsid. Typically, it may be a virus which is pathogenic to an animal, as defined herein, and may exclude plant viruses. Thus, the virus may be one which is not native to a plant cell, and therefore the viral saccharide will be heterologous to the plant cell. It may be produced by a virus-infected cell, using host cell machinery.

An immunogenic bacterial or viral saccharide is a saccharide which is capable of eliciting an immune response in a mammal, preferably a human. Bacterial immunogenic saccharides may be pneumococcal. Most preferably, the saccharide is native to Streptococcus pneumoniae.

In a second aspect, the invention provides a genetically modified plant cell capable of producing i) a bacterial capsular saccharide, ii) a bacterial membrane saccharide and/or iii) a bacterial or viral immunogenic saccharide, wherein the plant cell comprises a heterologous protein and/or nucleotide sequence which enables the production of the bacterial or viral saccharide in the host plant cell.

In an embodiment, the genetically modified plant cell comprises i) a bacterial capsular saccharide, ii) a bacterial membrane saccharide and/or iii) a bacterial or viral immunogenic saccharide.

In a third aspect, the invention provides a plant, a plant part or plant extract, plant tissue or callus comprising a genetically modified plant cell capable of producing i) a bacterial capsular saccharide, ii) a bacterial membrane saccharide and/or iii) a bacterial or viral immunogenic saccharide, wherein the plant cell comprises a heterologous protein and/or nucleotide sequence which enables the production of the bacterial or viral saccharide in the plant cell. The heterologous protein and/or nucleotide sequence may be as described herein.

In an embodiment, the plant, plant part or plant extract, plant tissue or callus may comprise a genetically modified plant cell comprising i) a bacterial capsular saccharide, ii) a bacterial membrane saccharide and/or iii) a bacterial or viral immunogenic saccharide. Also provided is an edible vaccine consisting of a plant, plant part or plant extract comprising a genetically modified plant cell comprising i) a bacterial capsular saccharide, ii) a bacterial membrane saccharide and/or iii) a bacterial or viral immunogenic saccharide.

In a fourth aspect, a plant product comprising i) a bacterial capsular saccharide, ii) a bacterial membrane saccharide and/or iii) a bacterial or viral immunogenic saccharide is provided. Preferably, the plant product is derived from a plant cell, a plant, plant tissue, a plant extract or callus as defined herein.

In a fifth aspect, a bacterial capsular saccharide, a bacterial membrane saccharide and/or a bacterial or viral immunogenic saccharide obtained by a method of the invention is provided. The saccharide may be extracted from the plant cell. It may be isolated. The saccharide may be purified, such that it is substantially free of plant material.

In a sixth aspect, the invention provides a saccharide based product comprising a plant cell, plant, plant part, plant extract, plant tissue, or callus, or extracted or purified saccharide, according to the invention. Preferably, the product is a vaccine.

In a further aspect, the invention provides the use of a plant, plant cell, plant part, plant extract, plant tissue, callus, plant derived product, and/or a bacterial capsular saccharide, a bacterial membrane saccharide and/or a bacterial or viral immunogenic saccharide, as herein described, as a medicament, a medical device (including an implant or surgical filler or a surgical thread), a vaccine, a textile, a lubricant, a cosmetic, a detergent, an injectable, feedstock, a health supplement, or a food additive (such as a gel or thickening agent).

In a further aspect the present invention provides a vector comprising a nucleic acid sequence which encodes an enzyme which enables the production of i) a bacterial capsular saccharide, ii) a bacterial membrane saccharide and/or iii) a bacterial or viral immunogenic saccharide in a host plant cell. The enzyme may be as defined herein. The saccharide may be as defined herein.

### DETAILED DESCRIPTION OF THE INVENTION

Herein, the terms “non-native” and “heterologous” are used interchangeably. The terms “non-native” or “heterologous” mean that the product (e.g. saccharide), reaction step or pathway referred to is not found in that cell in nature or is not found in a previously stably modified cell, for example an ancestor. Thus, the term “native” means that the product, reaction step or pathway referred to is in its natural form, i.e. where it would be found in nature without human intervention, or has been previously, stable modified. For example, a “native” reaction step or pathway as described herein means one as it is found in nature, for example with reference to the cell type or sequence of reactions. Thus, a native plant saccharide biosynthesis pathway or reaction step is one which found in a plant cell in nature, without intervention by man, or has been previously stably modified, for example in an ancestor cell. After manipulation of a pathway, as defined by the present invention, such as by addition or removal of a reaction step, it may be referred to then as being “non-native” or “heterologous”, because it is no longer as previously found in host cell. For the purpose of the present invention, “heterologous” or “non-native” or equivalent terminology does not include reaction steps or pathways which have been previously introduced into the cell, for example by infection, for example by a bacteria.

Thus, a bacterial or viral saccharide as defined herein may be referred to as being “heterologous” or “non-native” to the host plant cell, because it is present or is being produced in a host cell in which it would not normally be found or produced e.g. in nature. The product, reaction step or pathway may also be described as “exogenous” or “recombinant”.

By capsular is meant a polysaccharide which forms part of a bacterial capsule, which is provided outside of the outer membrane as a defense. Capsular saccharides may also be referred to in the art as K antigens. A bacterial capsular saccharide may be water soluble. It may be acidic. It may have a molecular weight of 100-2000 kDa. A bacterial capsular saccharide may be linear or branched in structure, and may comprise repeating units of from 1 to 6 monosaccharides. Bacterial capsular saccharides according to the present invention may be selected from Neisseria meningitides a (serogroups A, B, C, W135 or Y), Streptococcus pneumonia (serotypes 3, 4, 6B, 9V, 14, 18C, 19F or 23F), Streprococcus agalactiae (types Ia, Ib, IIm III, IV, V, VI, VII or VIII), Heamophilus influenzae (typeable strains: a, b, c, d, e or f), the capsular polysaccharide (Vi antigen) of Salmonella typhi, Staphylococcus aures (for example, serotypes 5 and S), Enterococcus faecalis or E. faecium (trisaccharide repeats), Yersinia enterocolitica, Escherichia coli (K antigens), Vibrio cholera, Klebsiella pneumonia and aerogenes etc). Other bacterial capsular polysaccharides include chondroitin and hyaluronic acid. Preferred saccharides include pneumococcal saccharides, in particular type 3 or type 4 capsular saccharide. Preferably, a capsular polysaccharide is a streptococcal capsular polysaccharide. Preferably, the capsular saccharide is native to Streptococcus pneumoniae, and is a Streptococcus pneumoniae type 3 or type 4 capsular polysaccharide.

Bacterial membrane saccharides include lipopolysaccharide, lipooligosaccharides, and exopolysaccharides. Membrane saccharides include Lipo-oligosaccharides (LOS) from from Neisseria meningitides a (serogroups A, B, C, W135 or Y), or Heamophilus influenzae (typeable strains: a, b, c, d, e or f), LPS from Pseudomonas aeruginosa, (for example isolated from PAOI, O5 serotype), LPS from Yersinia enterocolitica, Vibrio cholera, Salmonella typhi, and Klebsiella spp. In addition saccharides could include cell wall saccharides from Streptococcus pneumonia (serotypes 4, 6B, 9V, 14, 18C, 19F or 23F), Streprococcus agalactiae (types Ia, Ib, IIm III, IV, V, VI, VII or VIII), Staphylococcus aureus (for example, serotypes 5 and S), Enterococcus faecalis or E. faecium (trisaccharide repeats).

Immunogenic bacterial saccharides may be any saccharide which is capable of eliciting an immune response in a mammal, and is native to a virus or pathogenic bacterium, for example Staphylococcus, Streptococcus, Chlamydia, Burkholeria, Mycobacterium, Pseudomonas (e.g. Pseudomonas aeruginosa), Shigella, E. coli, Campylobacter, Salmonella (e.g. Salmonella enterica), Bortedella (e.g. Bortedella pertussis), Neisseria (e.g. meningitides) and Haemophilus (e.g. Haemophilus influenza). Preferably, the saccharide is native to Streptococcus. Most preferably, the saccharide is native to Streptococcus pneumoniae. Examples of immunogenic saccharides include pneumococcal capsule serotypes 3, 4, 6B, 9V, 14, 18C, 19F or 23F, capsular polysaccharides types Ia, Ib, IIm III, IV, V, VI, VII or VIII) from Streptococcus agalactiae capsule serotypes 5 and 8 from Staphylococcus aureus. Capsular polysaccharides from Neisseria meningitides a (serogroups A, B, C, W135 or Y), Heamophilus influenzae (typeable strains: a, b, c, d, e or f), the capsular polysaccharide (Vi antigen) of Salmonella typhi. Capsular polysaccharides from Escherichia coli (K antigens), Yersinia enterocolitica, Vibrio cholera, Klebsiella pneumonia and aerogenes. In addition immunogenic saccharides could be (LOS) from from Neisseria meningitides a (serogroups A, B, C, W135 or Y), or Heamophilus influenzae (typeable strains: a, b, c, d, e or f), LPS from Pseudomonas aeruginosa, (for example isolated from PAOI, 05 serotype), LPS from Yersinia enterocolitica, Vibrio cholera, and Klebsiella pneumonia and aerogenes. In addition immunogenic saccharides could include cell wall saccharides such as techoic acid or lipotechoic acid from Streptococcus pneumonia (serotypes 3, 4, 6B, 9V, 14, 18C, 19F or 23F), Streprococcus agalactiae (types Ia, Ib, IIm III, IV, V, VI, VII or VIII) or Staphylococcus aureus (for example, serotypes 5 and S).

For the purposes of the present invention, capsular or membrane saccharides do not include secreted saccharides, such as dextran, xanthum gum, pullulan, gellan gum, and welan gum.

Where the bacterial or viral saccharide is referred to as being immunogenic, this means that it is capable of evoking an immune response. It may also be referred to as being antigenic, or an antigen. Preferably, it is capable of evoking an immune response in an animal, including for example aqua culture organisms such as fish, birds, chickens, and mammals. More preferably, the bacterial or viral saccharide is one which is capable of evoking an immune response in a mammal, including a farm animal such as cattle or sheep, pets such as dogs, cats, or humans. Preferably, it is capable of evoking an immune response in an animal to which it is heterologous. For example, where the saccharide is bacterial in origin, it may be immunogenic to a mammal such as a human.

Immunogenic polysaccharides are found in many other pathogenic bacteria, such as pathogenic strains described above. The polysaccharide synthesis pathways for many of these organisms are well documented. Indeed attempts have been made to standardise the nomenclature of related genes from different species (see, for example, Reeves P. R., et al., Trends in Microbiology (1996), 4: 495-503).

The term “saccharide” is intended to include disaccharides, oligosaccharides and polysaccharides. Monosaccharide refers to one sugar residue. Oligo- and polysaccharides comprise two or more sugar residues. Typically, an oligosaccharide comprises 2 to 8 sugar residues, and a polysaccharide comprises 8 or more sugar residues, for example, 10, 12, 14, 16, 18, 20, 30-40, 50, 60, 70, 80, 90, 100 or more sugar residues. The sugar residues are glycosidically linked in branched or unbranched chains. In the art and in relation to the present invention, the terms “oligosaccharide” and “polysaccharide” are often used interchangeably to describe a polymer comprising up to 100 or more sugar residues. The saccharides may be up to 5, 10, 20, 30, 50, 70, 80, 90 or 100, 200, 300, 400, 500, 600, 700, 800, 900 or 1,000 kDa, or greater, in weight. A saccharide may be in a native form.

A saccharide may be naturally occurring or synthetic. The saccharide may be a homosaccharide or a heterosaccharide. It may be acidic (comprising carboxyl groups, phosphate groups or sulphuric ester groups) or non-acidic. Preferably it is acidic. The saccharide may be water soluble or insoluble, preferably the former. It may be branched or unbranched. The saccharide may be a bacterial saccharide, for example hyaluronic acid or chondroitin that closely resembles mammalian polysaccharides.

The saccharide may be native to a bacteria or virus which is not infectious to a plant cell, more specifically a plant cell of the host plant cell species.

The present invention is based upon utilising and adapting an existing plant saccharide biosynthesis pathway to produce a bacterial or viral saccharide, such as an immunogenic saccharide, in a host plant cell.

A biosynthesis pathway, as referred to herein, is a series of chemical reactions within the cell, in which a starting chemical compound (for example a monosaccharide) is modified by a series of reaction steps to produce a different chemical, for example an oligo- or poly-saccharide. Thus, it involves the step-by-step modification of an initial molecule to form another product, wherein each modification is carried out by an enzyme. A biosynthesis pathway according to the present invention may be all or part of a series of reaction steps starting from production of a monosaccharide to joining monosaccharides to form an oligo- or poly-saccharide, and modification of the oligo or poly-saccharide.

Reference herein to “a” step of a biosynthesis pathway includes a plurality of reaction steps from the biosynthesis pathway, i.e. one, two, three, four, five or more steps, any two or more of which may be sequential or non-sequential.

Many plant saccharides are known. For example, polysaccharides predominate the cell wall of plants. There are several classes of plant polysaccharides. Hemicelluloses include xylans, glucoronoxylans, arabinoxylans, arabinogalactns II, glucomannans, xyloglucans and galactomannans. Xylans contains (1,4) linked xylose residues. Glucomannans contain glucose and xylose linked by 1,4-glycosidic bonds. Xyloglucans contain a backbone of (1,4) linked glucose residues with xylose side chains, backbones comprising for example, galactose, fucose and arabinose side chains are possible. The hemicelluloses include the same monosaccharides that form type 3 polysaccharide. Pectins include polysaccharides rich in galacturonic acid, rhamnose, arabinose and galactose, such as polygalacturonans.

Numerous plant saccharide biosynthesis pathways have been identified. See, for example, WO03/026389 and U.S. Pat. No. 6,194,638. When comparing the biosynthesis pathways found in the host plant cell in which the polysaccharide is to be produced with the structure of the bacterial or viral saccharide and/or its biosynthesis pathway, it is possible to identify any reaction step which are either necessary or require down regulation, to allow the heterologous saccharide to be produced within the host plant cell. Once any reaction step has been identified, it is possible to identify a heterologous protein, gene and/or nucleotide sequence which can be introduced into a host plant cell to provide the reaction step and so enable synthesis of the heterologous saccharide. Thus, for example, a protein or nucleotide sequence introduced into a host plant cell may provide an enzyme which is capable of carrying out a biosynthetic step which is required in the bacterial or viral saccharide biosynthesis pathway but absent from the relevant plant saccharide biosynthesis pathway.

Where down regulation of a reaction step is desired, for example to down regulate a competing reaction step or pathway, a protein or nucleotide sequence may be introduced into the host plant cell which serves to knock out or down regulate the competing biosynthesis step. For example, a protein may inhibit a plant enzyme which mediates the competing step or may affect expression of the relevant plant enzyme. A nucleotide sequence may inhibit expression of an enzyme, for example through antisense or affecting expression of a relevant plant enzyme.

It is envisaged that within the scope of the present invention, two or more different heterologous proteins or nucleotide sequences may be introduced into a host plant cell to provide a reaction step which enables production of a bacterial or viral saccharide therein.

In addition, it is within the scope of the present invention that two or more different bacterial and/or viral saccharides selected from i) a bacterial capsular saccharide, ii) a bacterial membrane saccharide and/or iii) a bacterial or viral immunogenic saccharide may be produced within a host plant cell. Where two or more heterologous saccharides are produced in the host cell, these may all be bacterial, all viral, or a mixture of bacterial and viral, as desired.

The reaction step which is provided in the host plant cell to enable the production of of i) a bacterial capsular saccharide, ii) a bacterial membrane saccharide and/or iii) a bacterial or viral immunogenic saccharide using host plant cell machinery, and optionally host plant cell substrate, may be the generation of a precursor or intermediate from a substrate unit, or a step in the conjugation of a substrate unit (including a precursor or intermediate) to another unit in the formation of a polysaccharide chain. Examples of biosynthesis steps and the relevant enzymes typically required for production of bacterial saccharides and absent from plants include a glycosyltransferase or a glycosyl synthase enzyme, and a step mediated by such an enzyme. Examples of glycosyl transferase enzymes useful in the present invention include, but are not limited to: alpha-2,3/2,6/2,8-sialyl transferases, UDP-ManNAc transferases for example WecG or RffM, polysialyl transferase, NeuS, UDP-GlcNAc transferase, KfiA or heparosan synthase B, CMP-KDO transferase (KdsB or KpsU), UDP-GlcA transferase, GumK in xanthan synthesis (EC 2.4.1.17), UDP-GaINAc transferase, CgtA/NeuA1, and UDP-GIuA transferase involved in GAG polymerization. Examples of glycosyl synthase enzymes useful in the present invention include, but are not limited to: MurA and MurB (of the pathway which produces UDP-MurNAc), RffE/VVecB/NeuC (of the pathway which produces UDP-ManNAc), UDP-GlcNAc-4-epimerase (of the pathway which produces UDP-GaINAc, EC 5.1.3.7) and UDP-GlcNAc-6-dehydrogenase (of the pathway which produces UDP-GlcNAcA, EC 1.1.1.136).

A nucleotide sequence for introduction into a host plant cell to enable the production of i) a bacterial capsular saccharide, ii) a bacterial membrane saccharide and/or iii) a bacterial or viral immunogenic saccharide may encode an enzyme or protein required for the heterologous reaction step (for example as defined above)

A protein or nucleotide sequence for down regulating a native plant reaction step (for example a competing step) may be a regulator sequence, as defined herein, or may encode a regulatory protein, such as a transcription factor, or may encode a DNA or RNA antisense sequence. As such, a nucleotide sequence or its expression product can modify expression, amount and/or activity of a native saccharide biosynthesis enzyme in the host plant cell, to down regulate or otherwise alter its expression pattern, to optimise production of a bacterial or viral saccharide. Methods of function of such regulatory proteins, expression products and antisense will be known to persons skilled in the art.

The substrate for saccharide synthesis may be native to the host plant cell, or plant from which the cell is derived. Alternatively, it may be provided heterologously in the host plant cell. In the latter case, this may be achieved by providing in the plant cell an enzyme necessary for the production of the substrate in the plant cell. Such enzymes include but are not limited to, MurA and MurB (of the pathway which produces UDP-MurNAc), RffE/VVecB/NeuC (of the pathway which produces UDP-ManNAc), UDP-GlcNAc-4-epimerase (of the pathway which produces UDP-GaINAc, EC 5.1.3.7) and UDP-GlcNAc-6-dehydrogenase (of the pathway which produces UDP-GlcNAcA, EC 1.1.1.136). It is also envisaged that precursors or the saccharide substrates may be provided in the host plant cell, for example where one or more of these are limiting factors in the synthesis of bacterial or viral saccharides by the plant cell.

Herein, a substrate is any molecule which is acted upon by a saccharide biosynthesis enzyme. A substrate of saccharide synthesis includes monosaccharides, which are polymerised into longer chains to form oligo or polysaccharides. In some embodiments, a substrate may be a disaccharide. The term substrate herein can mean the saccharide units (e.g. monosaccharides), which are added to the growing chain of the polysaccharide. Examples of specific units include glucose, frustose, galactose, xylose and ribose; more specifically D-glucose and glucoronic acid. The term substrate may include modified forms of saccharide units, for example precursors and intermediates, e.g. UDP forms.

By way of example, the pneumococcal type 3 polysaccharide is composed of repeating D-glucose (Glc) and D-glucuronic acid (GlcA) units, as (1>4)-β-D-Glcp-(1>3)β-D-GlcAp-(1>4). At least two functions are necessary for production of the type 3 polysaccharide: synthesis of the precursors UDP-glucose (UDP-Glc) and UDP-glucuronic acid (UDP-GlcA) and their subsequent polymerisation. Plants synthesise UDP-Glc and UDP-GlcA within the endomembrane system, as substrates for extracellular enzymes involved in the synthesis of cell wall polysaccharides. Thus, comparison of the plant and bacterial saccharide biosynthesis pathways leads to identification of the pneumococcal cps3S gene that codes for the type 3 synthase, and is able to use plant UDP-glucose (UDP-Glc) and UDP-glucuronic acid (UDP-GlcA) in the production of type 3 capsular polysaccharide.

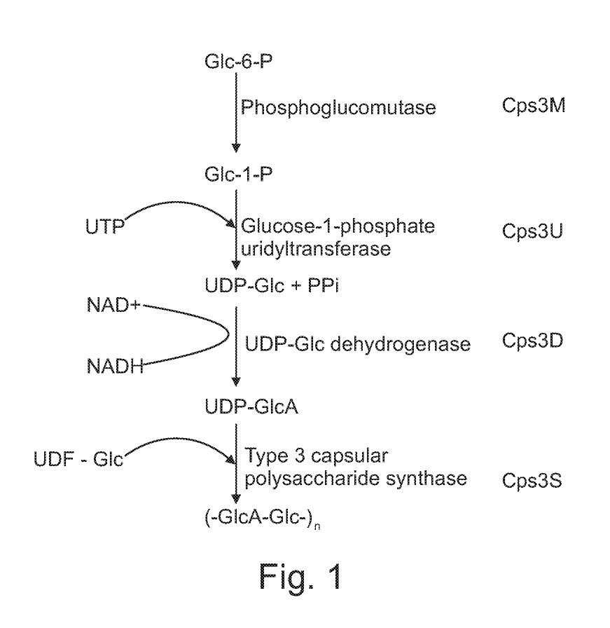

FIG. 1 shows the synthetic pathway of type 3 polysaccharide in pneumococci.

The genes involved in pneumococcal capsular polysaccharide synthesis are closely linked on the bacterial chromosome, arranged within a single locus (cassette) (Caimano et al. Capsule Genetics in Streptococcus pneumoniae and a possible role for transposition in the generation of the type 3 locus in Streptococcus pneumoniae. Edited by A. Tomasz. Larchmont, N.Y.: Mary Ann Liebert Inc. (2000)). These cassettes (cps) are termed “type-specific” because, although the genes involved in capsule synthesis of different serotypes share little homology, they may occupy identical sites in the chromosome (Caimano et al. (2000), Supra.). Furthermore genetic exchange of the type-specific cassettes results in transformation to the new type and the loss of the ability to express the original polysaccharide (Dillard et al. (1995), Journal of Experimental Medicine 181, 973-983). This implies that the expression of multiple pneumococcal genes could result in the production of all pneumococcal serotypes in planta. For in planta expression, each gene needs to be accompanied by a plant promoter (such as CaMV 35S promoter), yet this suggests that cloning a vector coding for all the genes represented in the type specific cassettes within a T-DNA region could result in the production of capsular polysaccharide from all 90 pneumococcal serotypes.

In an embodiment of the present invention, one or more of the CAP genes may be used to produce a bacterial saccharide in a plant cell plastid. The CAP genes include cap3A, cap3B and cap3C which encode, respectively UDP glucose dehydrogenase, type 3 polysaccharide synthase (cpS3S; Table 2) and glucose-1-phosphate uridyltransferase respectively, although others (including equivalents in other organisms) may be known to persons skilled in the art. In a preferred embodiment, two or more of the cap genes may be provided as a single operon. Most preferably, all three cap genes, cap3A, cap3B and cap3C are provided as a single operon. The operon may include a promoter, or may be targeted to a nuclear or plastid genome, and be expressed from a native promoter in the nucleus, cytoplasm or organelle.

Some genes and proteins required for pneumococcal saccharide production are summarised in Mavroidi et al; J. Bacteriol vol 89, No 21 7841-7855 (2007) and Table 1, although other genes and proteins which are available and not listed herein may also be used within the scope of the invention. The saccharide may be selected from a Streptococcal saccharide, such as one or more of the polysaccharides listed in Table 1 and Mavroidi et al. One or more of the genes listed above may be provided in the host plant cell, either as a nucleotide sequence for expression in the host plant cell or as an enzyme, to produce the saccharide.

The nucleotide sequence or protein required for the heterologous reaction step to enable production of the bacterial or viral saccharide in the plant cell may be bacterial or viral, or virus-infected cell in origin, but it is also envisaged that the nucleotide sequence or protein may be derived from other sources such as another plant, or a non-plant organism, such as a microbe (preferably a bacterium), a fungus, or an animal (an invertebrate such as insects or crustaceans, or vertebrate, such as a bird, sheep, cattle, dog, cat, or human). Typically, the nucleotide sequence or protein will be derived from a bacterial cell or a virus-infected cell. An example of a microbial heterologous gene is pneumococcal cps3S. Alternatively, other pneumococcal gene may be used, such as any of those described herein.

Nucleotide sequences encoding the enzymes or proteins for use in the invention will generally be available to those skilled in the art. The pneumococcal cps3S gene is available from Arrecubieta, C., Garcia, E. & Lopez, R. Demonstration of UDP-glucose dehydrogenase activity in cell extracts of Escherichia coli expressing the pneumococcal cap3A gene required for the synthesis of type 3 capsular polysaccharide. J. Bacteriol. 178, 2971-2974 (1996) and Dillard, J., Vandersea, M. & Yother, J. Characterization of the cassette containing genes for type 3 capsular polysaccharide biosynthesis in Streptococcus pneumoniae. J. Exp. Med. 181, 973-983 (1995)). The sequence of the type 3 pneumococcal capsular polysaccharide biosynthesis cassette is available from GenBank (www.ncbi.nlm.nih.gov), accession number U15171.1. Table 2 shows the amplified sequence:

atgt atacatttat tttaatgttg ttggattttt ttcagaatca
tgattttcat ttctttatgt tgttttttgt ctttattctt
attcgttggg cggttatata ttttcatgct gtcagatata
agtcctacag ttgtagtgta agtgatgaga agttatttag
ttctgtaatt atccctgtcg tggatgaacc acttaatctt
tttgaaagtg tactgaatag aatttccaga cataaaccat
ccgaaattat tgtggttatt aacggcccaa aaaacgagag
acttgtaaaa ctttgtcatg attttaatga aaaattagaa
aataatatga ctccaattca atgttattac actcctgttc
ctggcaagag aaatgctatc cgcgttgggc tggagcatgt
ggattcgcag agtgatatta cagttctagt agatagtgat
acagtatgga cgcctagaac cttgagtgag ttgctgaagc
cttttgtttg cgataaaaaa ataggtgggg taacgacaag
acaaaaaatt cttgaccctg agcgtaatct cgtgacaatg
tttgctaact tgttagagga aattagggca gaaggaacta
tgaaagcaat gagtgtgact ggtaaagtag ggtgcttacc
tggtcgaaca attgctttta gaaatatagt ggagagagtg
tatacaaagt ttatagaaga gactttcatg ggatttcata
aggaagtttc tgatgataga agtcttacaa atttgacttt
aaaaaaaggc tataaaactg ttatgcagga tacttctgtt
gtgtatacag atgctcctac aagttggaaa aagttcatta
gacagcaact aaggtgggca gaaggttctc agtataacaa
tctaaagatg actccttgga tgattagaaa tgcccctctt
atgtttttta tttattttac agatatgatt ttacctatgc
tacttattag ctttggtgtg aatatattcc tgttgaaaat
attaaatata actacaattg tttatacagc ttcatggtgg
gaaattattt tatatgttct tttgggaatg atttttagct
ttggaggaag aaactttaaa gctatgtcta gaatgaagtg
gtattatgta tttcttattc ctgtttttat aatcgttttg
agtataatta tgtgccctat taggctatta ggacttatga
gatgttctga tgatttaggg tggggaacta ggaatttaac
agagtga

Thus, a preferred heterologous saccharide biosynthesis gene may comprise the sequence of Table 2 or a fragment or derivative thereof.

Preferably, a nucleotide sequence used in the present invention will include an open reading frame encoding protein, and can further include non-coding regulatory sequences and introns. The term “nucleotide sequence” includes DNA molecules (e.g., a cDNA or genomic DNA) and RNA molecules (e.g., a mRNA) and analogs of the DNA or RNA generated, e.g., by the use of nucleotide analogs. The nucleic acid molecule can be single-stranded or double-stranded, but preferably is double-stranded DNA.

Also included within the present invention are fragments and derivatives of proteins or nucleotide sequences. Such fragments or derivatives may have one or more nucleotides or amino acids may be deleted or replaced, but share substantially the same functional characteristics as the unmodified protein or nucleotide sequence. Thus, it may still be capable of carrying out a saccharide biosynthesis reaction step as required. For example, a nucleotide sequence may encode or have deleted one or more amino acids at the N-terminal and/or C-terminal end without substantially altering the functionality of the expressed protein. Alternatively, or additionally, a sequence of the saccharide biosynthesis gene may be optimised for plant codon usage. Saccharide biosynthesis sequences may be optimised for increased expression in transformed plants. Methods are available for synthesising plant-preferred sequences, as demonstrated in, for example, U.S. Pat. No. 5,436,391 and U.S. Pat. No. 5,380,831.

Preferably a fragment or derivative of a nucleotide sequence or protein shares at least 70%, 75%, 80%, 85% or 90%, at least 91, 92, 93, 94, 95, 96, 97, 98, or at least 99% sequence identity with a reference nucleotide sequence or protein, over a length of 50%, 60%, 70%, 80%, 90%, or at least 95% of the length of a reference nucleotide sequence or protein.

The sequence may additionally be modified by, for example, elimination of sequences encoding sequences that may be deleterious to gene expression.

Sequence identity is determined by comparing the two aligned sequences over a pre-determined comparison window (which may be 50%, 60%, 70%, 80%, 90%, 95%, or 100% of the length of the reference nucleotide sequence or protein), and determining the number of positions at which -identical residues occur. Typically, this is expressed as a percentage. The measurement of sequence identity of a nucleotide sequences is a method well known to those skilled in the art, using computer implemented mathematical algorithms such as ALIGN (Version 2.0), GAP, BESTFIT, BLAST (Altschul et al J. Mol. Biol. 215: 403 (1990)), FASTA and TFASTA (Wisconsin Genetic Software Package Version 8, available from Genetics Computer Group, Accelrys Inc. San Diego, Calif.), and CLUSTAL (Higgins et al, Gene 73: 237-244 (1998)), using default parameters.

A nucleotide sequence may be provided as part of an expression cassette, for expression in a plant cell. Suitable expression cassettes may also comprise 5′ and 3′ regulatory sequences operably linked to the sequences of interest. The nature of any regulatory sequences provided in the expression construct will depend upon the desired expression pattern. Types of regulatory sequences will be known to persons skilled in the art. An expression cassette may also contain one or more restriction sites or homologous recombination sites, to enable insertion of the gene into the plant genome, at a pre-selected position. In the case of the nucleotide sequence, this may be operably linked to the gene sequence whose expression is to be modified. Also provided on the expression cassette may be transcription and translation initiation regions, to enable expression of the incoming genes, transcription and translational termination regions, and regulatory sequences. These sequences may be native to the host plant, or may be heterologous. An expression cassette may be a bi-functional expression cassette which functions in multiple hosts. A preferred expression cassette may be an Agrobacterium vector such as pCAMBIA 2301. For further details see, for example, Molecular Cloning: Laboratory Manual: 2^{nd} edition, Sambrook et al. 1989, Cold Spring Habor Laboratory Press or Current Protocols in Molecular Biology, Second Edition, Ausubel et al. Eds., John Wiley & Sons, 1992.

Thus, an expression cassette of the present invention may comprise one or more of:

a) a promoter;
b) a ribosome binding site;
c) a transcription termination region;
d) 3′ UTR e.g. for RNA stability
e) 5′ UTR e.g. for translation and for RNA stability
f) a selectable marker;
g) an origin of replication
f) a nucleotide sequence which enables insertion of the vector into the plant cell
genome;
g) a nucleotide sequence encoding a bacterial or viral saccharide biosynthesis enzyme.

Alternatively, an expression cassette according to the invention need not include a promoter or other regulatory sequence, particularly if the cassette is to be used to introduce the nucleic acid sequence(s) into a cell for recombination into the genome.

“Operably linked” means joined as part of the same nucleic acid molecule, suitably positioned and oriented for transcription to be initiated from the promoter. DNA operably linked to a promoter is “under transcriptional initiation regulation” of the promoter.

A regulatory sequence is a nucleotide sequence which is capable of influencing transcription or translation of a gene or gene product, for example in terms of initiation, rate, stability, downstream processing, and mobility. Examples of regulatory sequences include promoters, 5′ and 3′ UTR's, enhancers, transcription factor or protein binding sequences, start sites and termination sequences, ribozyme binding sites, recombination sites, polyadenylation sequences. The regulatory sequences may be plant- or virus derived, and preferably may be derived from the same species of plant as the plant cell in which the gene is to be expressed.

The expression cassette may also comprise elements such as introns, enhancers, and polyadenylation sequences, intergenic transcribed spacer elements, and RNA processing sites. These elements must be compatible with the remainder of the vector. These elements may not be necessary for the expression or function of the gene but may serve to improve expression or functioning of the nucleic acid sequence by affecting transcription, stability of the mRNA, or the like. Therefore, such elements may be included in the expression construct to obtain the optimal expression and function of the heterologous nucleic acid sequence.

By “promoter” is meant a nucleotide sequence upstream from the transcriptional initiation site and which contains all the regulatory regions required for transcription. Suitable promoters include constitutive, tissue-specific, inducible, developmental or other promoters for expression in a plant cell. A promoter may be viral, fungal, bacterial, animal or plant-derived, and capable of directing gene expression in a plant cell. A promoter is preferably tissue or organ specific, such that expression of a gene can be directed to a particular cell type of a plant. The promoter may also be developmental stage specific.

Examples of suitable promoter sequences include those of the T-DNA of A. tumefaciens, including mannopine synthase, nopaline synthase, and octopine synthase; alcohol dehydrogenase promoter from corn; light inducible promoters such as ribulose-biphosphate-carboxylase small subunit gene from various species and the major chlorophyll a/b binding protein gene promoter; histone promoters (EP 507 698), actin promoters; maize ubiquitin 1 promoter (Christensen et al. (1996) Transgenic Res. 5:213); 35S and 19S promoters of cauliflower mosaic virus; developmentally regulated promoters such as the waxy, zein, or bronze promoters from maize; as well as synthetic or other natural promoters including those promoters exhibiting organ specific expression or expression at specific development stage(s) of the plant, like the alpha-tubulin promoter disclosed in U.S. Pat. No. 5,635,618.

Reporter genes which encode easily assayable marker proteins are well known in the art. In general, a reporter gene is a gene which is not present or expressed by the recipient organism or tissue and which encodes a protein whose expression is manifested by some easily detectable property, e.g. phenotypic change or enzymatic activity. A selectable marker or reporter gene may be carried on a separate expression cassette and co-transformed with an expression cassette as described herein. A selectable marker and/or reporter gene may be flanked with appropriate regulatory sequence(s) to enable expression in a plant cell.

Preferred genes include the chloramphenicol acetyl transferase (cat) gene from Tn9 of E. coli, the beta-gluronidase (gus) gene of the uidA locus of E. coli, the green fluorescence protein (GFP) gene from Aequoria victoria, and the luciferase (luc) gene from the firefly Photinus pyralis. If desired, selectable genetic markers may be included in the construct, such as those that confer selectable phenotypes such as resistance to antibodies or herbicides (e.g. kanamycin, hygromycin, phosphinotricin, chlorsulfuron, methotrexate, gentamycin, spectinomycin, streptomycin, spectinomycin, phophinothricin, imidazolinones and glyphosate). Alternative selectable markers not based on antibiotics or herbicides may be included, for example those that result in restoration of photosynthesis when a lesion in the recipient plant cell is rescued by using a wild type gene for transformation. An example is rescue of the deleted rbcL gene with the wild type gene. The D-amino oxidase gene from Schizosaccharomyces pombe is an alternative marker gene that confers tolerance to D-amino acids such as D-alanine.

An expression cassette comprising a nucleotide sequence may comprise a sequence coding for a transit peptide, to drive a protein coded by the nucleotide sequence to a most suitable part of a plant cell such as the endoplasmic reticulum. Such transit peptides are well known to those of ordinary skill in the art, and may include single transit peptides, as well as multiple transit peptides obtained by the combination of sequences coding for at least two transit peptides. One preferred transit peptide is that from pathogenesis related protein of tobacco (PR1 b). This sequence directs the encoded protein to the endoplasmic reticulum of a plant cell.

An expression cassette may be a bi-functional expression cassette which functions in multiple hosts. In the case of GTase genomic DNA this may contain its own promoter or other regulatory elements and in the case of cDNA this may be under the control of an appropriate promoter or other regulatory elements for expression in the host cell.

Examples of a suitable expression cassette include an Agrobacterium vector, for example pCAMBIA 2301 or pART7.

Any suitable origin or replication or other sequences that promote autonomous replication in plastids may be used. These include sequences that allow maintenance through recombination with the main nuclear or plastid genome.

Suitable termination sequences for use with the heterologous gene include termination regions available from the Ti-plasmid of Agrobacterium tumefaciens, for example the well known octopine synthase and nopaline synthase termination regions.

In the present invention, any plant species may be used, including both monocots and dicots. Preferred plants for use in the present invention are those which are readily grown, exhibit high growth rates, are easily harvested, and are edible. Preferred plants include grasses, trees, crops, shrubs.

Preferably, plants of the present invention are crop plants (for example, cereals and pulses), maize, wheat, rye, potatoes, sweet potato tapioca, rice, sorghum, millet, cassava, barley, pea, tea, coffee, cassava, flax, canola, tobacco, cocoa, and other root, tuber or seed crops. Important seed crops are oil-seed rape, cotton, safflower sugar beet, maize, sunflower, soybean, alfalfa, palm, coconut and sorghum. Beans include guar, locust bean, fenugreek, soybean, garden beans, cowpea, mung bean, lima bean, fava bean, lentils, and chickpea. Leaf crops include lettuce and tobacco. Horticultural plants to which the present invention may be applied may include lettuce, endive, and vegetable brassicas including cabbage, tomato, broccoli, and cauliflower, and carnations, geraniums and chrysanthemum. Nuts include peanuts, cashew, macadamia, and almond. Fruits include strawberry, tomato, fig, guava, mango, papaya, avocado, banana, pineapple. Vegetables include pepper, olive, cucurbits and carrot.

Plants or plant cells transformed with a heterologous biosynthesis gene or expression construct of the invention may be produced by standard techniques known in the art for the genetic manipulation of plants Preferably the heterologous biosynthesis gene is incorporated using Agrobacterium tumefaciens-mediated gene transfer. Microprojectile bombardment, microinjection, electroporation, liposome-mediated DNA uptake or the vortexing method and direct DNA uptake amongst others are preferred where Agrobacterium is inefficient or ineffective. Alternatively, a combination of different techniques may be employed to enhance the efficiency of the transformation process, e.g. bombardment with Agrobacterium-coated microparticles (EP-A-486234) or microprojectile bombardment to induce wounding followed by co-cultivation with Agrobacterium (EP-A-486233).

A heterologous protein may be introduced into the host plant by any suitable means such as spraying, uptake by the roots, or injection into phloem.

A plant cell may be a part of a plant, a specific part of the plant or tissue, such as stem, leaves, root or tubers of the plant, fibres, seeds, biomass or fruit of the plant. A cell also may be a part of a callus or other undifferentiated mass of cells or alternatively a cell may be grown in culture medium.

A heterologous nucleotide sequence may be stably or transiently expressed in a plant cell. The nucleotide sequence is preferably stably integrated into the genome of the plant cell. However, it may also be transiently integrated into the cell and transiently expressed.

A plant cell, plant, plant tissue, plant extract, or callus, according to the invention, may be dried prior to use. Drying provides a way of conveniently storing the plant material comprising the heterologous saccharide prior to use. The dried material itself may be ground and sprinkled upon food stuffs as a way of conveniently in taking the saccharide, for example where the polysaccharide is intended to be a vaccine against, for example, pathogenic bacterium. Dried plant tissue may also provide a way of inoculating a patient. For example, the dried plant material may be taken through the nose in the form of snuff.

Plant extracts made by the methods of the invention or from plant cells, plants, plant tissue or calluses, according to the invention, and which comprise the heterologous polysaccharide, may simply be a crude extract, for example an aqueous extract in the form of a “tea” or an alcoholic extract. Alternatively, the saccharide may be further purified using methods well known in the art. The present invention provides the use of a plant part, plant tissue or plant extract as a vaccine, for the prevention of disease. Thus, the invention provides an edible vaccine comprising a modified plant cell of the invention and/or a bacterial or viral saccharide of the invention.

The saccharide of the present invention may extracted from a plant cell, meaning that it is removed from the cell. It may be purified. By “purified” is meant that it is substantially free from other cellular components or material, or culture medium. “Isolated” means that the saccharide may be free of naturally occurring sequences which flank the native sequence, for example in the case of nucleic acid molecule, isolated may mean that it is free of 5′ and 3′ regulatory sequences.

Where the glycosylated target is purified from a plant cell or plastid, it may be preferable to purify the glycosylated proteins by purification methods that are well known in the art. This includes affinity, size exclusion, ion exchange chromatography and ultracentrifugation. Purification of chloroplasts prior to fractionation based on physical properties of the glycosylated carrier can also be carried out.

The saccharide produced according to the invention may be conjugated, for example to produce a vaccine. Conjugation may take place within the host plant cell, or may be carried out after harvesting of the saccharide from the plant cell.

Vaccines of the invention may be administered via any suitable route, such as orally, or through the nose. Purified material may be injected, for example intrapareterally or subcutaneously.

A typical dose for a human would be in the range of 1-25 μg, preferably 1-10 μg. In a preferred embodiment, plastids are transformed with high copy numbers of heterologous nucleic acid to maximise the dose per gram of plant material.

The present invention has the advantage that a bacterial or viral saccharide as defined herein can be provided to a recipient by ingestion of plant material, without requiring further downstream processing. However, in certain circumstances, it may be desirable to extract and purify a bacterial or viral saccharide from plant material, and provide it to a recipient with a suitable excipient, diluent or adjuvant, which are well known in the art. An excipient or diluent is a solid or semi solid or liquid material which serves as a medium or vehicle for the active ingredient, in this case a bacterial or viral saccharide. A person of skill in the art will be capable of selecting an appropriate form and mode of administration for any particular product and condition. This is determined by the solubility and chemical properties of the bacterial or viral saccharide, the route of administration, and standard pharmaceutical practice. The preparation may be adapted for oral, parenteral or topical use, and may be administered in the form of tablets, capsules, suppositories, solutions, suspensions and the like. A bacterial or viral saccharide of the invention may be adsorbed to an aluminium salt adjuvant (e.g. a hydroxide or a phosphate), or may be unadsorbed (e.g. free in solution).

The features and embodiments of each aspect applies to the other aspects of the invention, mutatis mutandis.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The invention will now be described by way of example only with reference to the following figures and non-limiting examples:

FIG. 1: Proposed biosynthetic pathway of type 3 polysaccharide.

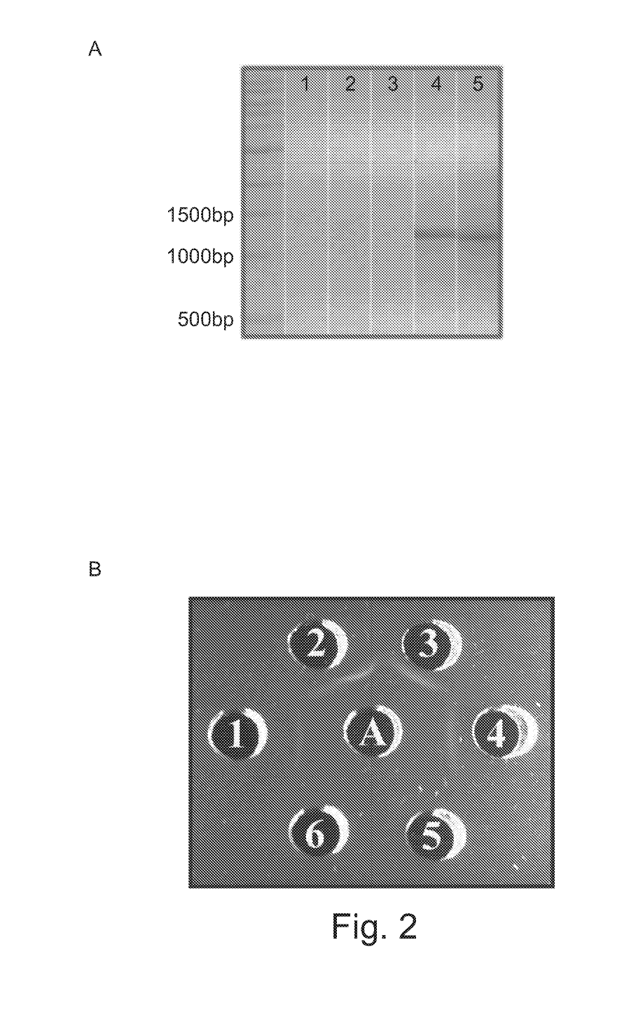

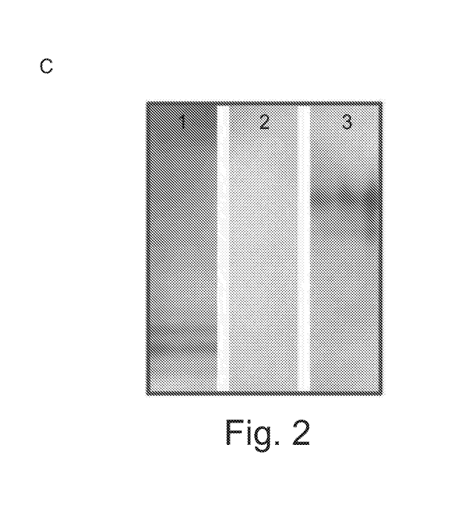

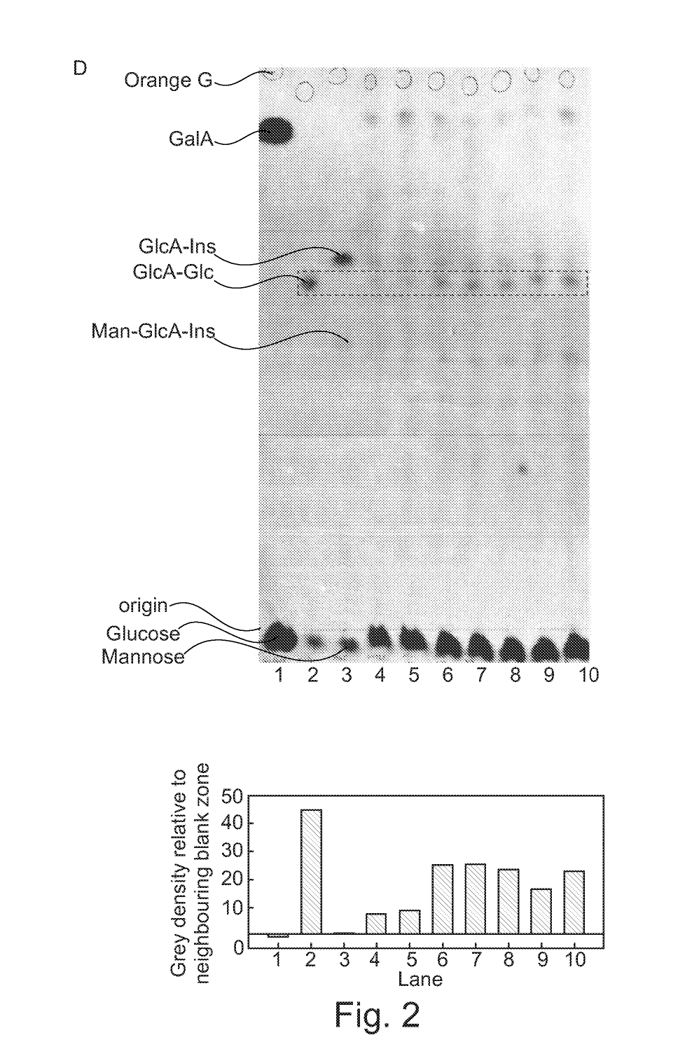

FIG. 2. In planta expression of the cps3S gene and formation of serotype 3 polysaccharide.

A. Reverse transcriptase PCR to detect cps3S mRNA in transgenic tobacco plants. RNA was extracted from a wildtype (Lanes 1 and 3) and a transgenic N. tabacum containing cps3S (Lanes 2 and 4). PCR, using cps3S specific primers, (Lanes 1 and 2) showed the absence of cps3S DNA in the RNA. RT-PCR on the same samples showed the presence of cps3S mRNA in the transgenic plant (Lane 4) but not in the wildtype (Lane 3). Lane 5 PCR of pCMS4 containing cps3S, done as before. The 1.25 kb amplicon in Lanes 4 and 5 shows a full-length transcript of cps3S is expressed in the transgenic plant.
B. Ouchterlony immuno-double diffusion. Well 1: 10 μg purified serotype 3 polysaccharide; Wells 2-4: extract from tobacco plants shown to express cps3S: Wells 5 and 6: extract from a wildtype tobacco plant. Well A: type 3 polysaccharide specific antiserum. The preciptin lines identify the presence of type 3 polysaccharide.
C. Western blotting using type 3 polysaccharide specific antiserum. Lane 1: purified type 3 polysaccharide; Lane 2: wildtype plant extract; Lane 3: transgenic plant extract.
D. High-voltage paper electrophoresis of tobacco leaf acid hydrolysates. Lanes 1-3: 25 μg of each marker, (Lane 1) galacturonic acid (GalA) and glucose, (Lane 2) glucose and β-D-glucuronosyl-(1→4)-D-glucose (GlcA-Glc) (partial hydrolysate of 10 μg type 3 pneumococcal polysaccharide) and (Lane 3) 10 μg of a mixture of mannose, α-D-glucuronosyl-(1→2)-myo-inositol (GlcA-Ins) and a trace of α-D-mannosyl-(1→4)-α-D-glucuronosyl-(1→2)-myo-inositol (Man-GlcA-Ins). Lanes 4-10: hydrolysate of polysaccharides cold-acid-extracted from 32 mg fresh weight of wildtype (Lanes 4 and 5) or transgenic (Lanes 6-10) tobacco leaves. Each lane also contains a trace of Orange G (coloured internal marker). All lanes show similar levels of staining for neutral sugars (co-migrating with glucose, near the origin). The samples were electrophoresed¹¹ at pH 6.5, at 3.0 kV for 60 min (anode at top) and stained with AgNO₃. Spots of the disaccharide, GlcA-Glc, diagnostic of type 3 pneumococcal polysaccharide, are highlighted by the dashed box; these spots were quantified for grey density in PhotoShop (see histogram).

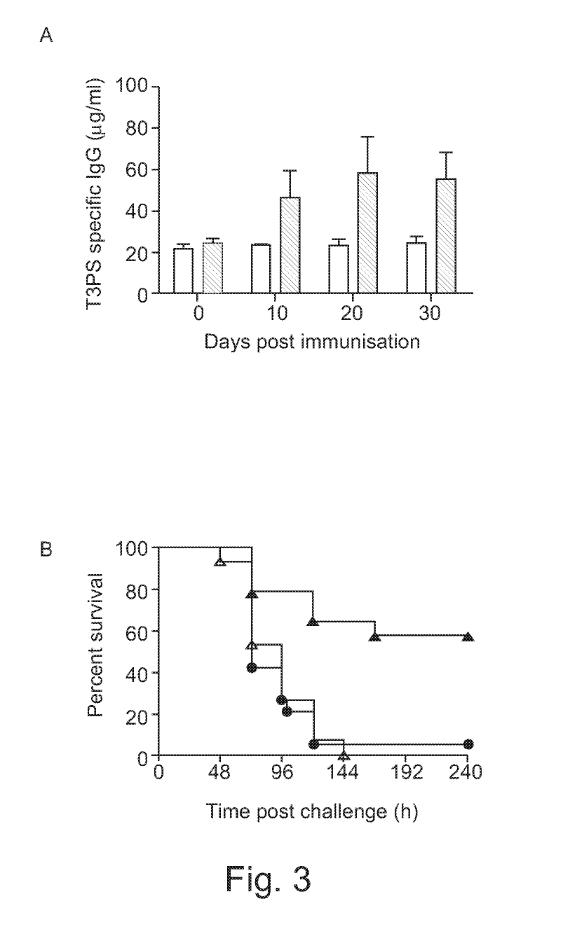

FIG. 3. Immunogenicity and protective efficacy of serotype 3 pneumococcal polysaccharide produced in planta.

A. Concentration of serotype 3 polysaccharide-specific IgG in serum of mice immunised with extracts from tobacco plants expressing cps3S (black bars) or wildtype plant (white bars); n=5.
B. Survival of mice challenged with virulent type 3 pneumococci 260 days after immunisation with transgenic plant extract (closed triangles), wildtype extracts (open triangles) or sham-immunised mice (closed circles). Mice alive at 240 h post-infection were considered to have survived the infection.

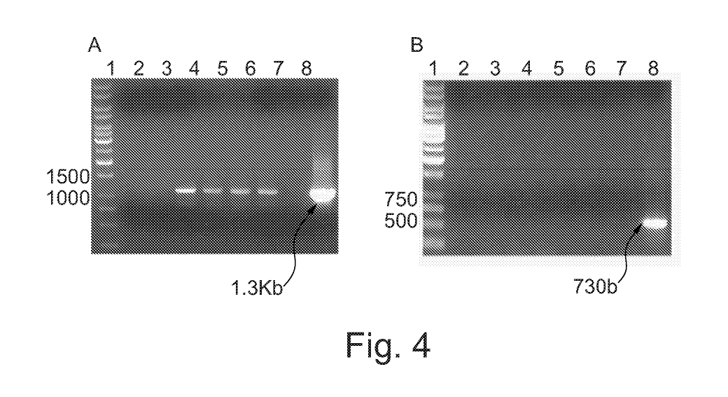

FIG. 4: Detection of the cps3S gene in transformed tobacco plants. DNA was extracted from the same six N. tabacum plants for the PCRs shown in A and B.

A. DNA was used as a template for PCR (Lanes 1, 2: wild type plants; Lanes 3-6: transformed plants.) using cps3S-specific primers. The results show the presence of the cps3S gene in the transformed plants (Lanes 3-6) but not the wild type plants. The PCR reaction in Lane 8 contained purified plasmid DNA containing cps3S (pCMS4) as a positive control and Lane 7 contained no template DNA. Molecular sizes are indicated. B. PCR showing the absence of Agrobacterium DNA contaminating DNA preparations from wild type (Lanes 1, 2) and transformed (Lanes 3-6) tobacco plants. PCR was done with Agrobacterium-specific primers. The results show that there was no Agrobacterium DNA present in the transgenic plant samples. The PCR reaction in Lane 8 contained Agrobacterium DNA as a positive control and shows the expected 730 bp band and Lane 7 contained no template. Molecular sizes are indicated.

### EXAMPLES

### Construction of Plant Expression Vector

The sequence of the type 3 pneumococcal capsular polysaccharide biosynthesis cassette was obtained from GenBank (www.ncbi.nlm.nih.gov), accession number U15171. A 1.3 kb DNA fragment containing the cps3S gene was obtained by PCR from the genomic DNA of S. pneumoniae serotype 3 (WU2) using the oligonucleotide primers sense (CPSFOR) 5′-CTG GTA↓CCC ATG TAT ACA TTT ATT TTA ATG TTG TTG G-3′ (SEQ ID NO 1) corresponding to 2227 bp-2254 bp with a KpnI restriction site inserted at the 5′ end; anti-sense (CPSREV) 5′-TCA TCA CTC TGT TAA ATT CCT AGT TCC-3′ (SEQ ID NO 2), corresponding to 3454 bp-3477 bp of the cassette. The amplified fragment was inserted into the multiple cloning site of pCR4-TOPO (Invitrogen, Carlsbad, Calif., USA). The Agrobacterium binary vector pPZP221 (Hajdukiewicz, et al. (1994), Plant Molec. Biol., 25: 989-994) has been previously engineered to produce vector pCHF2 containing the constitutively expressed cauliflower mosaic virus (CaMV) 35S promoter and a rbcS terminator (C. Fankhauser, personal communication). Here, synthetic primers homologous to the PR1b signal peptide sequence (Lund and Dunsmuir (1992), Plant. Mol. Biol., 18: 47-53) were annealed, phosphorylated and inserted into pCHF2 using SacI and KpnI cutting sites situated between the CaMV 35S promoter and the rbcS terminator. The cps3S gene was removed from the pCR4-TOPO vector using KpnI and PstI and inserted between the PR1 b signal sequence and the rbcS terminator sequence, to form the clone pCMS3, The entire expression cassette was then excised from pCMS3 with EcoRI and HinDIII and ligated into the binary plant vector pCAMBIA2301 (CAMBIA, Canberra, Australia) (containing a kanamycin-resistance gene and gus) to give the resulting plasmid, pCMS4.

Plant Transformation pCMS4 was introduced into Agrobacterium tumefaciens strain GV3101 directly by the heat shock method, using 0.5 μl (1 μg) pCMS4 and 0.1 ml of frozen CaCl2- competent A. tumefaciens cells. Cells were thawed at 37° C. for 5 minutes, re-suspended in 1 ml of YEP broth (10 g/L Yeast Extract, 10 g/L Peptone, 5 g/L NaCl, pH 7.0) and incubated at 28° C. for 2 hours with gentle shaking. Cells were harvested by centrifugation at 600 g for 10 minutes, the pellet re-suspended in 0.1 ml YEP and spread onto a YEP agar containing 100 μg/ml kanamycin and 100 μg/ml rifampicin and incubated overnight at 28° C. Subsequently, A. tumefaciens carrying pCMS4 was used to transform tobacco (Nicotiana tabacum cv SR1) leaf discs, as described previously (Draper, et aL (1988), Plant genetic transformation and gene expression: a laboratory manual, Blackwell Scientific Publications, Oxford). Leaf discs were then transferred to Murashige-Skoog (MS) agar containing 3% (w/v) sucrose, the plate was sealed with parafilm and incubated in the dark at room temperature for 2 days. Discs were then transferred to selective medium (MS agar containing 3% (w/v) sucrose, 250 μg/ml cefotaxime (to kill the A. tumefaciens on the surface of explants), 100 μg/ml kanamycin, 100 μg/ml ampicillin, 100 μg/ml naphthalene acetic acid (NAA) and 1 mg/ml 6benzylaminopurine) to select transgenic progeny. Ten days post infection, those discs showing shoot or callus formation were transferred to fresh selective medium and re-incubated for approximately 1-2 weeks. Larger shoots were transferred to powder rounds containing the same medium and incubated for a further 3 weeks, or until roots started to form. After eight weeks, kanamycin-resistant shoot regenerants were removed to a rooting medium containing 100 μg/ml NAA and 25 mg/L kanamycin. Rooted plantlets were transferred to soil, self-pollinated and the seeds stored desiccated at 4° C.
Detection of cps3S Gene Expression in Transgenic Plants (See FIG. 2).

Total RNA was isolated from 100 mg of leaf tissue using a RNA isolation kit (RNeasy Plant Mini Kit; Qiagen, Surrey, UK) and used in the production of first strand cDNA using a cDNA synthesis kit (RNase H reverse transcriptase kit; Invitrogen) and a random hexanucleotide primer. PCR was then performed using the CPSFOR and CPSREV primers as described above, RNA extracts without prior reverse transcriptase treatment were used as a control to indicate the presence of cps3S specific DNA. RNA extracted from wild-type tobacco leaves also was used as negative control. To confirm the absence of contaminating Agrobacterium DNA PCR was done with the primers VCF (5′-ATC ATT TGT AGC GAC T-3′ (SEQ ID NO 3)) and VCR (5′-AGC TCA AAC CTG CTT C-3′ (SEQ ID NO 4)), designed to amplify a 730 bp region of the virC gene of agrobacterial Ti and Ri plasmids (Sawada, et al (1995), PCR detection of Ti and Ri plasmids from phytopathogenic Agrobacterium strains, 61: 828-831).

### Preparation of Leaf Extracts.

Leaves were collected from tobacco plants, tissue ground under liquid nitrogen and nanopure water added to give 0.5 g/ml plant tissue. The cells were lysed by sonication: 6×30 second sonications at an amplitude of 50 microns with 30 second rests in between sonications. The cell lysate then was centrifuged for 5 minutes at 10,000 g and the supernatant divided into 1 ml volumes, lyophilised and stored at 4° C.

Extraction of Type 3 Polysaccharide from the Apoplastic Fluid.

A modification of the method described by Fry and co-workers was used (Fry, S. (1988), The Growing Plant Cell Wall: Chemical and Metabolic Analysis, John Wiley and Sons, New York). Leaf material (1 g) was added to 50 ml of 50 mM CaCl2 and vacuum-infiltrated for a period of 30 minutes. The leaves were removed and dried gently on a paper towel before being transferred to the barrel of a 25 ml syringe with the plunger removed. This was placed in a 50 ml Falcon tube and the assembly centrifuged at 800 g at 10° C. for 10 minutes. The aqueous extract was stored at 4° C. until required.

### Ouchterlony Immunodiffusion.

A modification of the method described by Ouchterlony and Nilsson ((1973), Immunodiffusion and immunoelectrophoresis, Blackwell Scientific Publications, Oxford) was used. 0.2% w/v Ouchterlony agarose in barbitone buffer (1.84 g/I diethylbarbituric acid, 10.3 g/I sodium diethylbarbiturate, pH8.6) was used to coat microscope slides. These were left to dry for 1 hour, and then overlaid with 4.5 ml 1% (w/v) agarose in barbitone buffer. Once set, 4 mm holes were cut and 20 μl of sample was placed in the outer holes. Type 3 polysaccharide from S. pneumoniae (ATCC), diluted in 20 μl of normal plant extract, was used as a positive control. The central hole contained 20 μl neat rabbit anti-type 3 polysaccharide antiserum (Statens Serum Institute, Copenhagen, Denmark). The slides were incubated at 4° C. in a humidity box for 1-2 weeks. Precipitin lines were observed and photographed in indirect light. Recombinant pneumococcal polysaccharides were estimated by comparison with the intensity of the precipitin lines of the positive controls.

High-Voltage Paper Electrophoresis (HYPE).

Leaf material was harvested, washed, cut into pieces and ground into a fine powder under liquid nitrogen. Samples were stored at −20° C. until required. To 10 g fresh weight was added 50 ml 5% (v/v) formic acid and the suspension was incubated with gentle shaking for 2 days at room temperature. This procedure is expected to extract the capsular polysaccharide, but only a small proportion of the leaf cell-wall polysaccharides and starch. The homogenate was filtered through Miracloth and rinsed with 25 ml water and then the combined filtrate was adjusted to pH 4.0 with pyridine. Co-extracted proteins were denatured at 100° C. for 60 min, then cooled and pelleted by centrifugation at 1700×g for 15 min; the supernatant was freeze-dried. The dried material was washed exhaustively at room temperature in several changes of 82.6% (v/v) ethanol, which dissolves low-MW sugars. The remaining insoluble, polysaccharide-rich material was then air-dried, incubated at 90° C. for 30 min in 22.6 ml water and cooled; trifluoroacetic acid (TFA) was then added to a final concentration of 0.36 M, which solubilised the polysaccharide.

For partial hydrolysis of the polysaccharide to yield the relatively acid-resistant, diagnostic dimer (aldobiouronic acid; GlcA-Glc) a portion of the solution was hydrolysed in 2 M TFA at 120° C. for 30 min [conditions optimised in preliminary runs with authentic type 3 polysaccharide; data not shown], then dried in vacuo. The hydrolysis products were redissolved in water containing a trace of Orange G (internal anionic marker), and a volume (equivalent to 32 mg fresh weight of leaf) was spotted on to Whatman 3MM paper. The samples were subjected to HVPE at pH 6.5, at 3.0 kV for 60 min (Fry, S. C. High-voltage paper electrophoresis (HVPE) of cell-wall building blocks and their metabolic precursors (Springer, New York, High-voltage paper electrophoresis (HVPE) of cell-wall building blocks and their metabolic precursors. In The Plant Cell Wall Methods and Protocols. Z. A. Popper, Ed., Springer, New York, pp. 55-80 (2011) and then stained with silver nitrate (Fry, S. C. The Growing Plant Cell Wall: Chemical and Metabolic Analysis. The Blackburn Press, Caldwell, N.J. (2000). to reveal sugars. External markers, run on the same sheet, included hydrolysates of (i) purified Type 3 polysaccharide (yielding glucose plus GlcA-Glc), and (ii) the trimer α-D-mannosyl-(1→4)-α-D-glucuronosyl-(1→2)-myo-inositol (Smith, C. K., Hewage, C. M., Fry, S. C. & Sadler, I. H. a-d-Mannopyranosyl-(1→4)-a-d-glucuronopyranosyl-(1->2)-myo-inositol, a new and unusual oligosaccharide from cultured rose cells. Phytochemistry 52, 387-396 (1999) which yields a comparable dimer, α-D-glucuronosyl-(1→2)-myo-inositol, plus mannose). Other markers were commercial glucose and galacturonic acid. After staining with silver nitrate (Fry, S. C. The Growing Plant Cell Wall: Chemical and Metabolic Analysis. The Blackburn Press, Caldwell, N.J. (2000), electrophoretograms were scanned and relevant spots were quantified for grey density in PhotoShop as described in Supplementary FIG. 1 of Parsons et al (Alternative pathways of dehydroascorbic acid degradation in vitro and in plant cell cultures: novel insights into vitamin C catabolism Biochem. J. 440, 375-383 (2011).

### Immunisation and Challenge.

Nine-week-old MF1 female mice (HarlanOlac, Bicester, UK) were given three doses of plant extract containing 2 μg plant-derived pneumococcal polysaccharide per mouse (as estimated by the Ouchterlony method) in 67 μl PBS and 33 μl Imject Alum adjuvant (Pierce, Rockford, Ill., USA). Mice were immunised intraperitoneally on days 0, 10, 20 and 30. Shamimmunised mice received Alum adjuvant containing an irrelevant immunogen (KLH) using the same schedule. Serum samples were obtained by tail bleeding the day before each immunisation. Mice were challenged intraperitoneally with 2.8×10⁶ cfu serotype 3 pneumococci on Day 260. The health status of animals was monitored, according to the scheme of Morton et al. ((1985)m Vet Record, 116: 431-436). These experiments were done under a project license from the UK Home Office.

### ELISA.

Maxisorb ELISA wells (Gibco BRL, Nunc products) were coated with 2 μg/ml purified type 3 pneumococcal polysaccharide (ATCC) in coating buffer (50 nM NaHCO3 pH9.6, 0.02% (w/v) NaN3) for 16 h at 22° C. After rinsing with PBS the wells were blocked with PBS+5% (w/v) Marvel at 37° C. for 1 h and washed three times with washing buffer (50 mM TrisHCl pH7.5, 150 mM NaCl, 0.05% (v/v) Tween20). Mouse sera were diluted 1:100 in blocking buffer and 100 μl added to the wells and incubated, shaking, for 2 hours at 37° C. The plates were washed three times as before and bound antibodies were detected using alkaline phosphatase-conjugated goat anti-mouse IgG secondary antibody (Fc specific, Sigma; diluted 1:5000), and 1 mg/ml p-nitrophenyl phosphate (Sigma) dissolved in 1 M diethanolamine pH 9.8, 0.5 M MgCl2. Absorbance was read at 405 nm after 1 hour at 37° C. and IgG concentration determined by reference to a standard curve prepared with murine IgG (Statens Serum Institute).

### DETAILED DISCUSSION

The cps3S gene was amplified from the DNA of pneumococcal strain WU2 and cloned into the Agrobacterium binary vector pCambia 2301 (CAMBIA, Canberra, Australia) to give pCMS4. This cloning placed cps3S under the control of duplicated constitutive cauliflower mosaic virus promoters, CaMV35S. It also provided a signal sequence from the tobacco pathogenesis-related protein, PR1 b, to direct the transgene product via the endoplasmic reticulum to the apoplast. The vector also enabled the subsequent selection of transformed plants with kanamycin. Sequencing of cps3S in pCMS4 showed it to be identical to the previously published sequence (Arrecubieta, et al. Gene 167, 1-7 (1996)).

Plasmid pCMS4 was successfully cloned into Nicotiana tabacum by Agrobacterium tumefaciens-mediated gene transfer. A T1 generation was grown from the seed of six plants and PCR was used to show that four of these contained the cps3S gene but not A. tumefaciens DNA (see FIG. 1). RT-PCR, with cps3S-specific primers, showed that the transgene was expressed in the transgenic plants (FIG. 3A shows the data from one transgenic plant). No PCR product was generated from the direct PCR amplification of RNA extracts, confirming the absence of any contaminating cps3S DNA (FIG. 3A). The absence of contaminating Agrobacterium DNA also was confirmed by PCR (see FIG. 3B). All four plants were shown to synthesise type 3 polysaccharide by Ouchterlony immunoassay of extracts of leaf tissue (see FIG. 3B shows the results from three of the plants). Growth of a second generation confirmed stable expression of the transgene (data not shown). Although no attempt was made to maximise expression of cps3S and synthesis of polysaccharide, based on the Ouchterlony data the yield of type 3 polysaccharide was estimated to be approximately 4 μg/g of leaf.

To test the immunogenicity of plant-derived pneumococcal type 3 polysaccharide, mice were immunised intraperitoneally with three doses of crude apoplast extracts from transgenic or wild type tobacco plants. Sera were collected on the day before each immunisation, and ten days after the final dose, and anti-type 3 polysaccharide IgG determined by ELISA. Significantly more (P<0.05) anti-type 3 polysaccharide IgG were detected after a single dose of the extract from a transgenic leaf compared with wild-type leaf extract (FIG. 4A). There was a further increase (P<0.05) in the concentration of anti-type 3 antibodies after a second dose of transgenic leaf extract. Two hundred and thirty days after the final immunisation dose the mice were challenged intranasally with the serotype 3 S. pneumoniae strain HB565. Mice were observed for up to ten days for the development of signs of disease. Mice immunised with transgenic plant extract survived significantly longer (P<0.001) than mice given extracts of the wild type plants (mean survival of 181 h±72 and 90 h±23 for the transgenic and wildtype, respectively). The mice infected with the wild type plant extract did not survive longer (P>0.05) than sham-immunised mice (mean survival of 91 h±38). None of the fifteen animals immunised with extract of wild type plants were alive ten days after the challenge whereas eight of the fourteen immunised with transgenic plant extract survived to the end of the study (FIG. 4B).

In summary, the data shown here prove the principle that bacterial polysaccharides can be synthesised in plants and that simple extracts of these plants can be used as immunogens to protect against otherwise lethal infection.

TABLE 1
Genes Required for Pneumococcal Polysaccharide Production

| | Danish Serotype | | |
|---|---|---|---|
| PNEUMOCCOAL | A: Primary Structures* | | |
| SEROTYPES | B: Enzyme(s) Required for Capsule Synthesis | | Reference |
| 1 | A | →3)-AAT-α-D-Galp-(1→4)-α-D-GalpA-(1→3)-α-D-GalpA-(1→ | Jiang et al, 2001 |
| | | +0.3 OAc | |
| | B | capA, capB, capC, capD, capE, capF, capG, capH, capI, capK | |
| 2 | A | 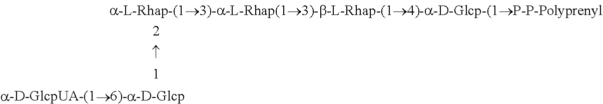 | Cartee et al, 2005 |
| | B | cps2A, cps2B, cps2C, cps2D, cps2E | |
| 3 | A | →4)-β-D-Glcp-(1→3)-β-D-GlcAp-(1→ | Arrecubieta et al, |
| | B | cps3U, cps3M, cps3S, cps3D | 1995; Dillard et al, |
| | | | 1995; This study |
| 4 | A | →3)-β-D-ManpNAc-(1→3)-α-L-FucpNAc-(1→3)-α-D-GalpNAc-(1→4)-α-D-Galp2,3(S)Pyr-(1→ | Jiang et al, 2001 |
| | B | wzg, wzh, wzd, wze, wciZ, wciJ, wciX, wciL, wzy, wciX, wzx, mnsA, orf13, orf14, orf15 | |
| 6A | A | →2)-α-D-Galp-(1→3)-α-D-Glcp-(1→3)-α-L-Rhap-(1→3)-D-Rib-ol-(5→P→ | |
| | B | Unknown | |
| 6B | A | →2)-α-D-Galp-(1→3)-α-D-Glcp-(1→3)-α-L-Rhap-(1→4)-D-Rib-ol-(5→P→ | Jiang et al, 2001 |
| | B | wzg, wzh, wzd, wze, wchA, wciN, wciO, wciP, wzy, wciX, wzx, xmlA, zmlC, rmlB, rmlD | |
| 8 | A | →4)-β-D-GlcpA-(1→4)-β-D-Glcp-(1→4)-α-D-Glcp-(1→4)-α-D-Galp-(1→ | Jiang et al, 2001 |
| | B | wzg, wzh, wzd, wze, wchA, wciQ, wciR, wciS, vzx, wciT, vzy, ugd | |
| 9A | A | →4)-α-D-GlcpA-(1→3)-α-D-Galp-(1→3) β-D-ManpNAc-(1→4)-β-D-Glcp-(1→4)-α-D-Glcp-(1→ | Bentley et al, 2006 |
| | B | dexB, tnp, wzg, wzh, wzd, wze, wchA, wchO, wcjA, mnaA, wzy, wcjB, wzx, wcjC, wcjO, tnp, ugd, | |
| | | wcjE, tnp, aftA | |
| 9L | A | →4)-α-D-GlcpA-(1→3)-α-D-Galp-(1→3) β-D-ManpNAc-(1→4)-β-D-Glcp-(1→4)-α-D-GlcpNAc- | Bentley et al, 2006 |
| | | (1→ | |
| | B | dexB, tnp, wzg, wzh, wzd, wze, wchA, wchO, wcjA, mnaA, wzy, wcjB, wzx, (twcjC), ugd, wcjE, tnp, | |
| | | aftA | |
| 9N | A | →4)-α-D-GlcpA-(1→3)-α-D-Glcp-(1→3) β-D-ManpNAc-(1→4)-β-D-Glcp-(1→4)-α-D-GlcpNAc- | Bentley et al, 2006 |
| | | (1→ | |
| | B | dexB, tnp, wzg, wzh, wzd, wze, wchA, wchO, (wcjA), mnaA, wzy, wcjB, wzx, (twcjC), ugd, wcjE, tnp, | |
| | | aftA | |
| 9V | A | 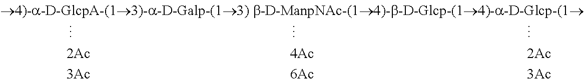 | Bentley et al, 2006 |
| | B | dexB, tnp, wzg, wzh, wzd, wze, wchA, wchO, wcjA, mnaA, wzy, wcjB, wzx, twcjC, wcjD, tnp, ugd, | |
| | | wcjE, tnp, aftA | |
| 12A | A | 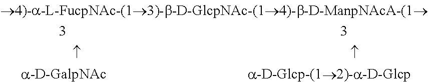 | Bentley et al, 2006 |
| | B | dexB, tnp, wzg, wzh, wzd, wze, tnp, wcif, wciJ, wcxB, wzy, wcxD, wcxE, wcxF, wzx, mnaB, mnaA, | |
| | | fnlA, fnlB, fnlC, tnp, aftA | |
| 12B | A | No Information | Bentley et al, 2006 |
| | B | dexB, tnp, wzg, wzh, wzd, wze, wcif, wciJ, wcxB, wzy, wcxD, wcxE, wcxF, wzx, mnaB, mnaA, fnlA, | |
| | | fnlB, fnlC, tnp, aftA | |
| 12F | A | 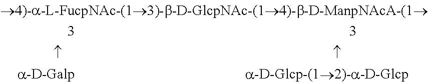 | Bentley et al, 2006; Kamerling, 2000 |
| | B | dexB, tnp, wzg, wzh, wzd, wze, wcif, wciJ, wcxB, wzy, wcxD, wcxE, wcxF, wzx, mnaB, mnaA, fnlA, | |
| | | fnlB, fnlC, tnp, aftA | |
| 14 | A | 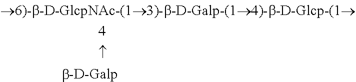 | Bentley et al, 2006 |
| | B | dexB, tnp, wzg, wzh, wzd, wze, wchA, wchJ, wchK, wzy, wchL, wchM, wchN, wzx, wciY, trp, tnp, | |
| | | aliA | |
| 15A | A | 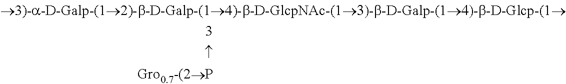 | Bentley et al, 2006 |
| | B | dexB, tnp, wzg, wzh, wzd, wze, wchA, wchJ, wchK, wzy, wchL, wchM, wchN, wzx, wciZ, wchX, gtp1, | |
| | | gtp2, gtp3, tnp, aliA | |
| 15B | A | 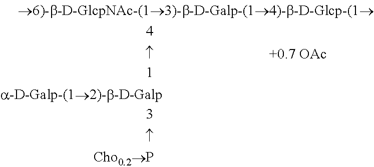 | Bentley et al, 2006 |
| | B | dexB, tnp, wzg, wzh, wzd, wze, wchA, wchJ, wchK, wzy, wchL, wchM, wchN, wzx, wciZ, wchX, | |
| | | gtp1, gtp2, gtp3, tnp, aliA | |
| 15C | A | 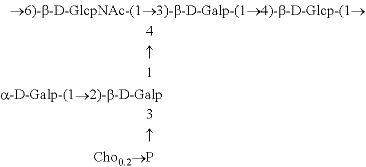 | Bentley et al, 2006 |
| | B | dexB, tnp, wzg, wzh, wzd, wze, wchA, wchJ, wchK, wzy, wchL, wchM, wchN, wzx, wciZ, wchX, gtp1, | |
| | | gtp2, gtp3, tnp, aliA | |
| 15F | A | 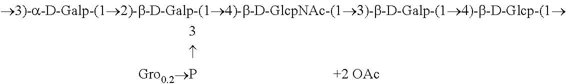 | Bentley et al, 2006 |
| | B | dexB, tnp, wzg, wzh, wzd, wze, wchA, wchJ, wchK, wzy, wchL, wchM, wchN, wzx, wciZ, wchX, gtp1, | |
| | | gtp2, gtp3, nntB, nntD, tnp, aliA | |
| 18C | A | 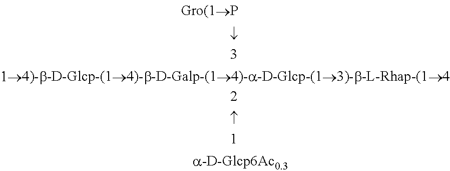 | Jiang et al, 2001 |
| | B | cpsA (wzg), cpsB (wzh), cpsC (wzd), cpsD (wze), cpsE (wchA), cpsF, cpsG, cpsH, cpsF, cpsI (wzy), | |
| | | cpsQ, cpsR, cpsJ (wzx), cpsK (mnaA), cpsL (rmlA), cpsM (rmlC), cpsN (rmlS), cpsO (rmlD) | |
| 19A | A | 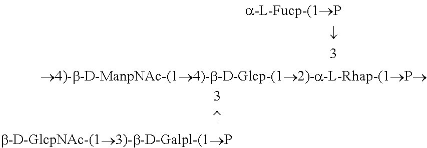 | Jiang et al, 2001 |
| | B | cpsA (wzg), cpsB (wzh), cpsC (wzd), cpsD (wze), cpsE (wchA), cpsF, cpsG, cpsH, cpsF, cpsI (wzy), | |
| | | cpsJ (wzX), cpsK (mnaA), cpsL (rmlA), cpsM (rmlC), cpsN (rmlS), cpsO (rmlD) | |
| 19B | A | 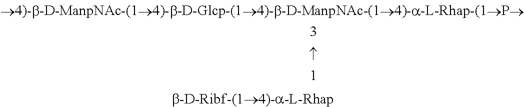 | Jiang et al, 2001 |
| | B | cpsA (wzg), cpsB (wzh), cpsC (wzd), cpsD (wze), cpsE (wchA), cpsF, cpsG, cpsH, cpsP, cpsI (wzy), | |
| | | cpsQ, cpsR, cspJ (wzx), cpsK (mnaA), cpsL (rmlA), cpsM (rmlC), cpsN (rmlS), cpsO (rmlD) | |
| 19C | A | 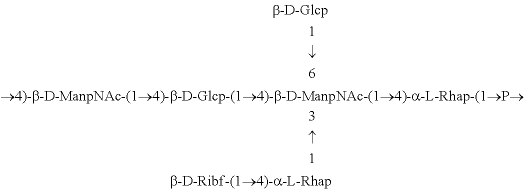 | Jiang et al, 2001 |
| | B | cpsA (wzg), cpsB (wzh), cpsC (wzd), cpsD (wze), cpsE (wchA), cpsF, cpsG, cpsH, cpsP, cpsI (wzy), | |
| | | cpsQ, cpsR, cspJ (wzx), cpsK (mnaA), cpsS, cpsL (rmlA), cpsM (rmlC), cpsN (rmlS), cpsO (rmlD) | |
| 19F | A | →4)-β-D-ManpNAc-(1→4)-α-D-Glcp-(1→2)-α-L-Rhap-(1→PO₄→ | Guidolin et al, 1994 |
| | B | dexB, IS1202, 19fA, 19fB, 19fC, 19fD, 19fE, F, G, H, I, J, K, L, M, N, O, plpA | |
| 23F | A | 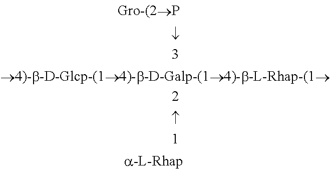 | Jiang et al, 2001 |
| | B | No Information | |
| 33F | A | 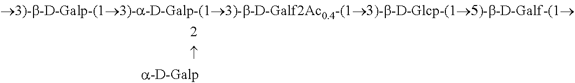 | Jiang et al, 2001 |
| | B | cpsA (wzg), cpsB (wzh), cpsC (wzd), cpsD (wze), cpsE (wchA), cpsT, cpsF (wzy), cpsU, cpsH, cpsV, | |
| | | cpsI, cpsJ (wzx), cpsW, cspK, cpsX, cpsL cpsY, cpsM, cpsZ, cpsN, cpsL, cpsO (rmlA), cpsM, cpsP | |
| | | (rmlC), cpsN, cpsQ (rmlS), cpsO, cpsR (rmlD) | |
| 44 | A | No Information | Bentley et al, 2006 |
| | B | dexB, tnp, wzg, wzh, wzd, wze, wcil, wciJ, wcxB , wzy, wcxD, wcxE, wcxF, mnaB, mnaA, flnA, fnlB, | |
| | | fnlC, tnp, aliA | |
| 46 | A | Constituents: D-GalNAc, D-GlcNAc, and L-FucNAc | Bentley et al, 2006 |
| | B | cpsA (wzg), cpsB (wzh), cpsC (wzd), cpsD (wze), cpsE (wchA), cpsF, cpsG, cpsH, cpsI, cpsJ, cspK | |
| | | (wzy), cpsL (wzx), cpsM, cpsN (gif), cpsO | |
| LT-B and ESAT-6 | A | A fusion of two immunogenic proteins that could enhance immune response | Rigano et al 2004 |
| | B | | |

*Taken from Kamerling, 2000

1. A method of producing a bacterial capsular saccharide, a bacterial membrane saccharide and/or a bacterial or viral immunogenic saccharide in a host plant cell, the method comprising:
1) providing a plant cell;
2) introducing into the plant cell a heterologous protein or nucleotide sequence which enables the production of the bacterial or viral saccharide in the plant cell;
3) maintaining the host plant cell under suitable conditions for saccharide biosynthesis; and optionally
4) harvesting the bacterial or viral saccharide from the host plant cell.

2. A method of producing a bacterial capsular saccharide, a bacterial membrane saccharide and/or a bacterial or viral immunogenic saccharide in a plant cell according to claim 1, further comprising the steps of:
(i) identifying the molecular structure of the bacterial or viral saccharide and/or elucidating the native bacterial or viral saccharide biosynthesis pathway;
(ii) comparing the molecular structure of the bacterial or viral saccharide and/or the native bacterial or viral saccharide biosynthesis pathway identified in i) with the molecular structure of the native plant saccharide and/or the native plant saccharide biosynthesis pathway of the host plant cell, to identify any reaction steps required in the native plant saccharide biosynthesis pathway to enable its use in production of a bacterial or viral saccharide; and
(iii) identifying a protein or nucleotide sequence which can provide the reaction step identified in ii) to enable the production of a bacterial or viral saccharide in the host plant cell.

3. A method according to claim 1, wherein the heterologous nucleotide sequence encodes a bacterial or viral enzyme, or a functional fragment or derivative thereof.

4. A method according to claim 1, wherein the method further comprises providing in the plant cell a protein or nucleotide sequence which inhibits or down regulates a native plant saccharide biosynthesis pathway or reaction step, to enable production of the bacterial or viral saccharide in the host plant cell.

5. A method according to claim 1, wherein the method further comprises providing in the host plant cell a protein or nucleotide sequence to enable the production of substrate for saccharide biosynthesis, wherein the substrate is heterologous to the host plant cell.

6. A method according to claim 1, wherein the saccharide is bacterial in origin, preferably streptococcal, more preferably pneumococcal.

7. A method according to claim 6 wherein the saccharide is a bacterial capsular polysaccharide, preferably type 3.

8. A method according to claim 1, wherein the heterologous saccharide biosynthesis gene is a pneumococcal polysaccharide synthesis gene.

9. A method according to claim 8 wherein the heterologous saccharide biosynthesis gene encodes a glycosyltransferase or a glycosyl synthase enzyme, or an enzyme listed in Table 1, or a cap3A, B, or C gene product (e.g Cps3S).

10. A method according to claim 1, wherein two or more heterologous saccharide biosynthesis genes are expressed in the plant cell.

11. A method according to claim 1, additionally comprising extracting the saccharide from the host plant cell.

12. A modified plant cell capable of producing a bacterial capsular saccharide, a bacterial membrane saccharide and/or a bacterial or viral immunogenic saccharide, wherein the plant cell comprises a heterologous protein and/or nucleotide sequence which enables the production of the bacterial or viral saccharide in the hose plant cell.

13. A modified plant cell according to claim 12, wherein the plant cell comprises a protein or nucleotide sequence which inhibits or down regulates a native plant saccharide biosynthesis pathway reaction step, to enable production of the bacterial or viral saccharide in the host plant cell.

14. A modified plant cell according to claim 12, wherein the plant cell comprises a protein or nucleotide sequence to enable the production of substrate for saccharide biosynthesis, wherein the substrate is heterologous to the host plant cell.

15. A plant cell according to claim 12, wherein the heterologous saccharide biosynthesis protein or nucleotide sequence or enzyme is plant, bacterial, archaeal, fungal, invertebrate or vertebrate in origin.

16. A plant cell according to claim 12, wherein the heterologous saccharide biosynthesis protein or nucleotide sequence is bacterial, viral, or derived from a virus-infected cell.

17. A plant cell according to claim 12, wherein the heterologous saccharide biosynthesis gene is a pneumococcal polysaccharide synthesis gene.

18. A plant cell according to claim 17 wherein the heterologous saccharide biosynthesis gene encodes a glycosyltransferase or a glycosyl synthase enzyme, or an enzyme listed in Table 1, or a cap3A, B, or C gene product (e.g Cps3S).

19. A plant cell according to claim 12, wherein the saccharide is bacterial in origin, preferably streptococcal, more preferably pneumococcal.

20. A plant cell according to claim 19 wherein saccharide a bacterial capsular polysaccharide, preferably type 3.

21. A plant, plant tissue or plant extract comprising a plant cell according to claim 12.

22. A vaccine, comprising a plant, plant tissue or plant extract according to claim 21, preferably an edible vaccine.

23. A dried plant, plant tissue, or plant extract callus comprising a plant cell according to claim 12.

24. A bacterial capsular saccharide, a bacterial membrane saccharide and/or a bacterial or viral immunogenic saccharide produced by a method according to claim 1.

25. A vaccine comprising a bacterial capsular saccharide, a bacterial membrane saccharide and/or a bacterial or viral immunogenic saccharide produced by a method according to claim 1.

26. A saccharide derived products selected from the group consisting of a medicament, a medical device (including an implant or surgical filler or a surgical thread), a vaccine, a textile, a lubricant, a cosmetic, a detergent, an injectable, feedstock, a health supplement, or a food additive (such as a gel or thickening agent), comprising a plant cell plant, plant, plant extract, or plant tissue, or saccharide according to claim 12.

27. A bacterial or viral saccharide produced by a method according to claim 1.


SEQUENCE LISTING (4 sequences)

SEQ ID NO: 1 - DNA, 37 bp, Artificial Sequence
  CPSFOR
ctggtacccatgtatacatttattttaatgttgttgg

SEQ ID NO: 2 - DNA, 27 bp, Artificial Sequence
  CPSREV
tcatcactctgttaaattcctagttcc

SEQ ID NO: 3 - DNA, 16 bp, Artificial Sequence
  VCF
atcatttgtagcgact

SEQ ID NO: 4 - DNA, 16 bp, Artificial Sequence
  VCR
agctcaaacctgcttc